(19) United States
(12) Patent Application Publication
(10) Pub. No.: US 2009/0075898 A1
(43) Pub. Date: Mar. 19, 2009

(54) Complement C3A Derived Peptides and Uses Thereof

(75) Inventors: Israel Pecht, Rehovot (IL); Anna Erdei, Budapest (HU)

Correspondence Address: ROTHWELL, FIGG, ERNST & MANBECK, P.C., 1425 K STREET, N.W., SUITE 800, WASHINGTON, DC, 20005, US

(73) Assignee: Yeda Research and Development Co. Ltd. at the Weizmann Institute of Science, Rehovot (IL)

(21) Appl. No.: 11/995,895

(22) PCT Filed: Jul. 27, 2006
(86) PCT No.: PCT/IL2006/000878

§ 371 Date: Jul. 29, 2008

Related U.S. Application Data
(60) Provisional application No. 60/702,627, filed on Jul. 27, 2005.
(60) Provisional application No. 60/776,668, filed on Feb. 27, 2006.

(51) Int. Cl. A61K 38/10; C07K 7/06; A61K 38/08; A61P 37/08

(52) U.S. Cl. 514/13; 530/328; 530/327; 514/16; 514/15; 514/14

(57) ABSTRACT

Peptides corresponding partially to positions 55-64 of the sequence of the complement component peptide C3a are capable of preventing and treating mast cell- and basophil-mediated disorders by inhibiting IgE- or IgG-mediated triggering and/or by inhibiting the FcεRI- and/or FcγR-induced secretory response, while obviating the anaphylatoxic response. These peptides are useful for prevention and/or treatment of allergic disorders where mucosal-type and/or serosal-type mast cells and/or basophils are involved such as asthma, allergic dermatosis, and gastrointestinal allergies.

### FIELD OF THE INVENTION

The present invention relates to peptides derived from the amino acid sequence of complement C3a and to their use in the prevention and treatment of allergic disorders mediated by mast cells or basophils, particularly pulmonary allergies such as asthma.

### BACKGROUND OF THE INVENTION

Mast cells and basophils play a central role in inflammatory and immediate hypersensitivity reactions. Clustering of the type 1 Fcε receptors (FcεRI) present in the plasma membranes of mast cells and basophils initiates a coupling cascade culminating in the secretion of inflammatory mediators including histamine, serotonin, proteases, leukotriens and several cytokines. The molecular mechanism of signal transduction initiated by FcεRI clustering has been intensively studied over the past few years. Lyn, a src family protein tyrosine kinase (PTK) interacts with the β subunit of the receptor complex and undergoes phosphorylation and activation as a result of FcεRI clustering. Recruitment of Lyn to the immunoreceptor tyrosine-based activation motif (ITAM)-phosphorylated receptor subunits results in activation of Syk PTK which in turn causes phospholipase C-γ (PLC-γ) activation, hydrolysis of phosphatidyl-inositide-4,5-bisphosphate (PIP2) and a transient rise in free cytosolic [Ca2+]i. This in turn induces activation of protein kinase C culminating eventually in mediator secretion.

Mast cell progenitors represent a single lineage, giving rise, upon migration into different tissues to two distinct phenotypes; the so-called serosal (connective tissue type) mastocytes residing in serosal cavities, in the skin and respiratory tract; and the mucosal type mast cells found mainly in the gastrointestinal mucosal surfaces. Nevertheless, mast cell tissue-dependent differentiation is reversible; fibroblast derived factors change mucosal type mast cells into serosal ones, while IL-3 favors the mucosal phenotype. Besides tissue distribution, life span and mediator content of their intracellular granules are also different. Both types express FcεRI on their cell membrane, clustering of which provokes the secretory response.

In contrast to the FcεRI-mediated triggering of mastocytes, the peptidergic pathway of mast cell activation only occurs in serosal mast cells. Serosal mast cells are experimentally modeled by rat peritoneal or human skin mast cells. The peptidergic stimulus is triggered by exposure to polyamines or cationic peptides such as substance P, or the complement activation products C3a and C5a (Mousli et al., Immunopharmacol. 27: 1-11, 1995). The latter complement-derived anaphylatoxins are among the most potent peptidergic activators of (serosal) mast cells' secretory response. In contrast, mucosal mast cells, such as the rat basophilic leukemia cell line (RBL-2H3) do not respond to such cationic peptides. It was demonstrated that C3a and some of its derivatives inhibit the IgE-mediated degranulation of RBL-2H3 cells, while C5a has no effect on this process (Erdei et al. Int. Immunol. 7: 1433-1439, 1995; Erdei et al. Immunol. Lett. 68: 79-82, 1999).

The C3a is not suitable for use as an anti-allergic drug because it is anaphylatoxic to serosal mast cells, i.e., it is capable of inducing mediator secretion from mast cells.

U.S. Pat. No. 6,682,740 to the applicants of the present invention discloses peptides corresponding partially or entirely to positions 50-77 of the sequence of human complement-derived peptide C3a and analogs thereof capable of inhibiting IgE-mediated triggering and/or the FcεRI-induced secretory response of mucosal mast cells.

There remains an unmet need for short peptides and compositions comprising same useful for preventing or treating allergic disorders associated with basophil and both serosal-type and mucosal-type mast cell mediated degranulation.

### SUMMARY OF THE INVENTION

It has now been found in accordance with the present invention that certain peptides derived from and corresponding partially to the amino acid sequence at positions 55-64 of human complement component C3a and analogs thereof are effective in inhibiting the FcεRI-induced secretory response of both mucosal-type and serosal-type mast cells and basophils and in alleviating the attendant symptoms, while being devoid of the anaphylatoxic effect of C3a.

It has now been discovered that the inhibitory effect of these peptides provides inhibition of proximal events to the FcεRI stimulus-response coupling cascade such as protein phosphorylation of the FcεRI β subunit and of the tyrosine kinase Lyn as well as inhibition of later events such as the transient rise in free cytosolic calcium ions.

It is now disclosed for the first time that the C3a derived peptides corresponding partially to the amino acid sequence at positions 55-64 of human complement component C3a and analogs thereof are capable of reducing passive systemic anaphylaxis and asthma symptoms in animal models.

The novel peptides disclosed in the present invention are derived from the known human complement component C3 a, which is a 77-mer peptide (SEQ ID NO:1) having the sequence:

Ser-Val-Gln-Leu-Thr-Glu-Lys-Arg-Met-Asp-Lys-Val-
Gly-Lys-Tyr-Pro-Lys-Glu-Leu-Arg-Lys-Cys-Cys-Glu-
Asp-Gly-Met-Arg-Glu-Asn-Pro-Met-Arg-Phe-Ser-Cys-
Gln-Arg-Arg-Thr-Arg-Phe-Ile-Ser-Leu-Gly-Glu-Ala-
Cys-Lys-Lys-Val-Phe-Leu-Asp-Cys-Cys-Asn-Tyr-Ile-
Thr-Glu-Leu-Arg-Arg-Gln-His-Ala-Arg-Ala-Ser-His-
Leu-Gly-Leu-Ala-Arg.

The present invention relates to peptides derived from and corresponding partially to the amino acid sequence of positions 55-64 of human complement peptide C3a having the sequence: Asp-Cys-Cys-Asn-Tyr-Ile-Thr-Glu-Leu-Arg set forth in SEQ ID NO:2, to analogs, chemical derivatives, and salts thereof, capable of inhibiting the secretory response of a cell, wherein the cell is a mucosal-type mast cell, a serosal-type mast cell and/or a basophil; and wherein the secretory response is induced by a stimulus selected from (i) IgE- or IgG-mediated triggering; and/or (ii) FcεRI or FcγR clustering.

In one aspect, the present invention provides a peptide derived from the sequence of amino acids 55-64 (SEQ ID NO:2) of human complement C3a having the amino acid sequence of general formula I:

X1-Asp-X2-Asn-Tyr-Ile-Thr-X3 (SEQ ID NOs:3 to 10);

wherein

X1 is selected from hydrogen, lower alkanoyl, Cys, Ser, D-Ala, and D-Ala-D-Ala;

X2 is selected from Ser-Ser and Val-Val; and

X3 is selected from Arg, Arg-NH2, Glu-Cys-Arg, and Glu-Cys-Arg-NH2;

or an analog, chemical derivative, or pharmaceutically acceptable salt thereof; with the proviso that when X1 is hydrogen or lower alkanoyl and X3 is Arg or Arg-NH2, then X2 is Val-Val.

The novel peptides of the present invention are capable of inhibiting a secretory response of a cell selected from the group consisting of a mucosal-type mast cell, a serosal-type mast cell and a basophil. The secretory response being induced by a stimulus selected from the group consisting of (i) IgE- or IgG-mediated triggering, and/or (ii) FcεRI or FcγR clustering.

In some embodiments, the present invention provides a peptide having an amino acid sequence selected from the group consisting of:

(SEQ ID NO:7)

(a) D-Ala-D-Ala-Asp-Ser-Ser-Asn-Tyr-Ile-Thr-Arg-Z;

(SEQ ID NO:11)

(b) Ser-Asp-Ser-Ser-Asn-Tyr-Ile-Thr-Arg-Z;

(SEQ ID NO:12)

(c) Asp-Val-Val-Asn-Tyr-Ile-Thr-Arg-Z;

(SEQ ID NO:13)

(d) Cys-Asp-Ser-Ser-Asn-Tyr-Ile-Thr-Arg-Z;

(SEQ ID NO:14)

(e) Ser-Asp-Ser-Ser-Asn-Tyr-Ile-Thr-Glu-Cys-Arg-Z;
(f) an analog of (a), (b), (c), (d) or (e);
(g) a chemical derivative of (a), (b), (c), (d),
(e), or (f);
and
(h) a salt of (a), (b), (c), (d) ,(e), (f), or
(g);

where Z designates a terminal carboxy acid, amide, or alcohol.

The term D-Ala refers to the D-isomer configuration of alanine.

According to some embodiments, the peptide of the invention has about 8 to about 12 amino acid residues. In specific embodiments, the peptide of the invention has about 8 to about 10 amino acid residues.

According to certain embodiment, the peptide has an amino acid sequence set forth in SEQ ID NO:7, wherein the Z is a carboxy terminal amide. According to certain embodiment, the peptide has an amino acid sequence set forth in SEQ ID NO:11, wherein the Z is a carboxy terminal amide.

According to some exemplary embodiments, the Z is an amide and the peptide is selected from the group consisting of:

(SEQ ID NO:7)

(a) D-Ala-D-Ala-Asp-Ser-Ser-Asn-Tyr-Ile-Thr-Arg-
NH2 denoted herein C3a32;

(SEQ ID NO:11)

(b) Ser-Asp-Ser-Ser-Asn-Tyr-Ile-Thr-Arg-NH2
denoted herein C3a31;

(SEQ ID NO:12)

(c) Asp-Val-Val-Asn-Tyr-Ile-Thr-Arg-NH2 denoted
herein C3a14;

(SEQ ID NO:13)

(d) Cys-Asp-Ser-Ser-Asn-Tyr-Ile-Thr-Arg-NH2
denoted herein C3a29;

(SEQ ID NO:14)

(e) Ser-Asp-Ser-Ser-Asn-Tyr-Ile-Thr-Glu-Cys-Arg-
NH2 denoted herein C3a35.

According to another aspect, the present invention provides a pharmaceutical composition comprising as an active agent at least one peptide derived from the sequence of amino acids 55-64 of human complement C3a, or an analog, chemical derivative, or a pharmaceutically acceptable salt thereof according to the principles of the present invention, and a pharmaceutically acceptable carrier. According to some embodiments, the peptide within the pharmaceutical composition has an amino acid sequence selected from the group consisting of SEQ ID NOs:3 to SEQ ID NO:14. In certain embodiments, the peptide within the pharmaceutical composition has an amino acid sequence set forth in SEQ ID NO:7, wherein the carboxy terminus is optionally a carboxy terminal amide. In additional embodiments, the peptide within the pharmaceutical composition has an amino acid sequence set forth in SEQ ID NO:11, wherein the carboxy terminus is optionally a carboxy terminal amide.

According to a further aspect, the present invention provides the use of at least one peptide, or an analog, chemical derivative, or a pharmaceutically acceptable salt thereof, and a pharmaceutically acceptable carrier for the preparation of a medicament useful in the prevention and/or treatment of an allergic disorder, wherein the peptide, analog, chemical derivative or salt thereof has an amino acid sequence selected from the group consisting of SEQ ID NOs:3 to SEQ ID NO:14. In certain embodiments, the peptide has an amino acid sequence set forth in SEQ ID NO:7 or SEQ ID NO:11, wherein the carboxy terminus is optionally a carboxy terminal amide. According to some embodiments, the allergic disorder is a basophil- and/or mucosal-type and/or serosal-type mast cell mediated disorder. According to an exemplary embodiment, the allergic disorder is asthma.

According to yet further aspect, the present invention provides a method for the prevention and/or treatment of an allergic disorder comprising administering to a subject in need thereof a therapeutically effective amount of a pharmaceutical composition comprising as an active agent a peptide having an amino acid sequence selected from the group consisting of SEQ ID NOs:3 to SEQ ID NO:14, or an analog, chemical derivative, or pharmaceutically acceptable salt thereof; and a pharmaceutically acceptable carrier, thereby preventing or treating the allergic disorder. According to certain embodiments, the peptide is selected from the group consisting of SEQ ID NOs:3 to 14. According to certain embodiments, the peptide has an amino acid sequence set forth in SEQ ID NO:7 or SEQ ID NO:11, wherein the carboxy terminus is optionally a carboxy terminal amide.

In some embodiments, the allergic disorder results from an IgE- or IgG-mediated (Type I or Type III) hypersensitivity and/or FcεRI- or FcγR-induced secretory response. According to other embodiments, the allergic disorder is mediated by a cell type selected from the group consisting of mucosal-type mast cells, serosal-type mast cells and basophils. Examples of allergic disorders that can be treated and/or prevented with the pharmaceutical compositions of the invention include, but are not limited to, allergic rhinitis, including seasonal rhinitis and sinusitis; pulmonary diseases such as asthma; allergic dermatosis such as urticaria, angioedema, eczema, atopic dermatitis, and contact dermatitis; allergic conjunctivitis; gastrointestinal allergies such as those caused by food or drugs; cramping; nausea; vomiting; diarrhea; irritable bowel disease; ophthalmic allergies; cheilitis; vulvitis; uveitis; and anaphylaxis. According to an exemplary embodiment, the allergic disorder is asthma.

According to another aspect, the present invention relates to a method for the prevention and/or treatment of an allergic disorder mediated by a cell type selected from the group consisting of serosal-type mast cells and basophils, the method comprising administering to a subject in need thereof a therapeutically effective amount of a pharmaceutical composition comprising as an active agent a peptide selected from the group consisting of the following sequences:

(SEQ ID NOs:15 to 20)

(a) X1-Cys-Asn-R1-X4;

(SEQ ID NOs:21 to 23)

(b) X2-Lys-Val-Phe-Leu-Asp-X3;
and

(SEQ ID NO:24)

(c) X5-Asp-Ser-Ser-Asn-Tyr-Ile-R7;

wherein

X1 is selected from hydrogen, lower alkanoyl, Cys, Asp-Cys and Arg-Arg-Cys;

X2 is selected from hydrogen, lower alkanoyl and Lys;

X3 is selected from

(i)
Ala-Ala-Asn-R1-Ile-Thr-R2-Leu-R3-R4;
(ii)
Cys-Cys-Asn-R1-Ile-Thr-R2-Leu-R3;
and
(iii)
Cys-Cys-Asn-R1-Ile-Thr-R2-Leu-R3-R4-Gln-His-R5-R6;

X4 is selected from Ile-Thr-R2-Leu-R3; and Ile-Thr-Arg-R7;

X5 is selected from lower alkanoyl and Leu;

R1 is selected from an aromatic amino acid residue;

R2 is selected from Glu and Lys;

R3 is selected from a positively charged amino acid residue;

R4 is selected from Arg and Glu;

R5 is selected from Ala and Arg;

R6 is selected from Arg and Lys;

R7 is selected from hydroxy (OH), Arg, Arg-NH2, and Agm (agmatine);

analogs, chemical derivatives and pharmaceutically acceptable salts thereof, and a pharmaceutically acceptable carrier.

In additional embodiments the peptide is selected from the group consisting of the following sequences:

(SEQ ID NO:25)

(a) Cys-Cys-Asn-Tyr-Ile-Thr-Glu-Leu-Arg;

(SEQ ID NO:26)

(b) Asp-Cys-Cys-Asn-Tyr-Ile-Thr-Arg;

(SEQ ID NO:27)

(c) Asp-Ser-Ser-Asn-Tyr-Ile-Arg denoted herein
C3a11;

(SEQ ID NO:28)

(d) Lys-Val-Phe-Leu-Asp-Cys-Cys-Asn-Tyr-Ile-Thr-
Glu-Leu-Arg denoted herein C3a4;

(SEQ ID NO:29)

(e) Lys-Lys-Val-Phe-Leu-Asp-Cys-Cys-Asn-Tyr-Ile-
Thr-Glu-Leu-Arg-Arg-Gln-His-Ala-Arg denoted herein
C3a5;

(SEQ ID NO:30)

(f) Lys-Val-Phe-Leu-Asp-Ala-Ala-Asn-Tyr-Ile-Thr-
Glu-Leu-Arg-Arg denoted herein C3a6;

(SEQ ID NO:31)

(g) Arg-Arg-Cys-Cys-Asn-Tyr-Ile-Thr-Arg-Arg
denoted herein C3a10;
(h) an analog of (a), (b), (c), (d), (e), (f) or
(g);
(i) a chemical derivative of (a), (b), (c), (d),
(e), (f), (g), or (h);
and
(j) a salt of (a), (b), (c), (d), (e), (f), (g),
(h), or (i).

In some embodiments the peptide has the amino acid sequence set forth in any one of SEQ ID NOs:25 to SEQ ID NO:31, wherein the carboxy terminus is optionally a carboxy terminal amide. In specific embodiments, the peptide is selected from the group consisting of the following sequences:

(SEQ ID NO:25)

(a) Cys-Cys-Asn-Tyr-Ile-Thr-Glu-Leu-Arg-NH2
denoted herein C3a7;

(SEQ ID NO:26)

(b) Asp-Cys-Cys-Asn-Tyr-Ile-Thr-Arg-NH2 denoted
herein C3a9;

In some embodiments, the allergic disorder to be treated and/or prevented with the pharmaceutical composition comprising a peptide selected from the group consisting of SEQ ID NOs: 15 to 31 results from an IgE- or IgG-mediated (Type I or Type III) hypersensitivity and/or FcεRI- or FcγR-induced secretory response in serosal-type mast cells and/or basophils. Examples of allergic disorders that can be treated include, but are not limited to, gastrointestinal allergies such as those caused by food or drugs; cramping; nausea; vomiting; diarrhea; and vulvitis. It is to be understood that U.S. Pat. No. 6,682,740 discloses methods for treating allergic disorders caused by IgE-mediated (Type I) hypersensitivity where mucosal-type mast cells are involved comprising administering to a subject in need thereof a peptide having the amino acid sequence of any one of SEQ ID NOs:15 to 31 or an analog or derivative thereof. The present invention discloses for the first time methods for treating or preventing allergic disorders where serosal-type mast cells and/or basophils are involved comprising administering to a subject in need thereof the peptide having the amino acid sequence of any one of SEQ ID NOs:15 to 31 or an analog or derivative thereof.

These and other embodiments of the present invention will be better understood in relation to the figures, description, examples and claims that follow.

### BRIEF DESCRIPTION OF THE DRAWINGS

FIGS. 1A-C illustrate the inhibitory capacity of synthetic peptides with sequences analogous to C3a stretches on the IgE-mediated secretory response of RBL-2H3 cells. FIG. 1A depicts the effect of different concentrations of C3a7 or C3a9 on the IgE mediated release of the granular enzyme, β-hexosaminidase from RBL-2H3 cells. FIG. 1B depicts the effect of 250 μg of C3a31, C3a32, or C3a35 on the IgE-mediated release of β-hexosaminidase from RBL-2H3 cells. FIG. 1C depicts the effect of C3a7 and C3a9 on the secretion of TNF-α cytokine by RBL-2H3.

FIG. 2A-B illustrate the inhibition of tyrosine phosphorylation of Lyn, β subunit of FcεRI and PI-3K by C3a7 and C3a9. FIG. 2A, general phosphorylation pattern of RBL-2H3 cells. FIG. 2B, Phosphorylation of Lyn, β-chain of FcεRI and PI-3K. Whole cell lysates were immunoblotted with anti-actin (bottom rows) to confirm that equal amounts of proteins were loaded.

FIG. 3 depicts the inhibition of antigen-induced rise of free cytosolic Ca2+ ions in mast cells by peptides C3a7 and C3a9.

FIGS. 4A-E depict the interaction of C3a with the β-chain of the high affinity IgE receptor. FIG. 4A, Detection of the covalent complex of C3a and β-chain of FcεRI on bone marrow derived mast cells by Western blotting with an antibody specific to the β-chain of FcεRI (lane 1). In control sample (lane 2) no C3a was present. FIGS. 4B and 4C, show results of Surface Plasmon Resonance (SPR) measurements: Biotinylated peptides representing the 1st extracellular loop of the rat (B) and human (C) FcεRI β chain were immobilized on the SPR-sensor chips and their interactions (i.e. association and dissociation) with C3a, used as analyte was followed in real time. FIG. 4D, Confocal microscopic images of RBL-2H3 cells fluorescently-labeled with Cy3-IgE and Cy5-C3a9. Equatorial slices (upper row) and a composite of three optical slices at the top of the cell (lower row) are shown for a representative cell. FIG. 4E, Histogram of fluorescence resonance energy transfer (FRET) efficiency between FITC-C3a9 (donor) and Cy3-IgE (acceptor) bound to RBL-2H3 cells.

FIG. 5 depicts the effect of C3a31 and its control reversed sequence peptide denoted C3a55 on blood histamine levels in mice exposed to passive systemic anaphylaxis.

FIG. 6 illustrates the protective capacity of C3a31 as compared to its control reverse peptide denoted C3a55 as measured by lung function in a murine asthma model.

### DETAILED DESCRIPTION OF THE INVENTION

The present invention relates to synthetic peptides based on the C-terminal sequence of the human complement C3a, analogs, chemical derivatives, and pharmaceutically acceptable salts thereof. The peptides are useful for inhibiting IgE- or IgG-mediated (Type I and Type III) hypersensitivity and/or FcεRI- or FcγR-induced secretory response, wherein the response is mediated by mast cells of both the mucosal and serosal-type and basophils.

The peptides of the invention are derived from and corresponding partially to the amino acid sequence at positions 55-64 of the human complement C3a set forth in SEQ ID NO:2 as follows:

Asp-Cys-Cys-Asn-Tyr-Ile-Thr-Glu-Leu-Arg

and to analogs, chemical derivative and pharmaceutically acceptable salts thereof. The C-terminus of the peptides of the invention can be in its free carboxy form or, preferably, it can be amidated to increase the stability of the peptide, e.g., to increase the resistance of the peptide to enzymatic cleavage in the organism. The carboxy terminus can also be modified in a way that increases its solubility.

### Peptides of the Present Invention

The present invention provides novel peptides useful in inhibiting the FcεRI- or FcγR-induced secretory response and/or IgE or IgG-mediated (Type I or Type III) mediated hypersensitivity of mast cells and basophils. The mast cells include mucosal type and serosal type mast cells.

According to one aspect, the present invention provides a peptide derived from the sequence of amino acids 55-64 (SEQ ID NO:2) of human complement C3a consisting of the amino acid sequence of general formula I:

X1-Asp-X2-Asn-Tyr-Ile-Thr-X3 (SEQ ID NOs:3 to 10);

wherein

X1 is selected from hydrogen, lower alkanoyl, Cys, Ser, D-Ala, and D-Ala-D-Ala;

X2 is selected from Ser-Ser and Val-Val; and

X3 is selected from Arg, Arg-NH2, Glu-Cys-Arg, and Glu-Cys-Arg-NH2;

or an analog, chemical derivative, or pharmaceutically acceptable salt thereof; with the proviso that when X1 is hydrogen or lower alkanoyl and X3 is Arg or Arg-NH2, then X2 is Val-Val.

In some embodiments, the present invention provides a peptide consisting of an amino acid sequence selected from the group consisting of:

(SEQ ID NO:7)

(a) D-Ala-D-Ala-Asp-Ser-Ser-Asn-Tyr-Ile-Thr-Arg-Z;

(SEQ ID NO:11)

(b) Ser-Asp-Ser-Ser-Asn-Tyr-Ile-Thr-Arg-Z;

(SEQ ID NO:12)

(c) Asp-Val-Val-Asn-Tyr-Ile-Thr-Arg-Z;

(SEQ ID NO:13)

(d) Cys-Asp-Ser-Ser-Asn-Tyr-Ile-Thr-Arg-Z;

(SEQ ID NO:14)

(e) Ser-Asp-Ser-Ser-Asn-Tyr-Ile-Thr-Glu-Cys-Arg-Z;
(f) an analog of (a), (b), (c), (d) or (e);
(g) a chemical derivative of (a), (b), (c), (d),
(e), or (f);
and
(h) a salt of (a), (b), (c), (d) ,(e), (f), or
(g);

where Z designates a terminal carboxy acid, amide, or alcohol.

The term D-Ala refers to the D-isomer configuration of alanine.

In one embodiment, the peptide of the invention is the peptide herein identified as peptide C3a32 set forth in SEQ ID NO:7, a 10-mer peptide derived from the 53-62 sequence of human complement peptide C3a, where the carboxy terminus is a carboxy terminal amide, of the sequence:

D-Ala-D-Ala-Asp-Ser-Ser-Asn-Tyr-Ile-Thr-Arg-NH2

In another embodiment, the peptide of the invention is the peptide herein identified as peptide C3a31 set forth in SEQ ID NO:11, a 9-mer peptide derived from the 54-62 sequence of human complement peptide C3a, where the carboxy terminus is a carboxy terminal amide, of the sequence:

Ser-Asp-Ser-Ser-Asn-Tyr-Ile-Thr-Arg-NH2

In still further embodiments, the peptides of the invention include an 8-mer peptide denoted herein C3a14, a 9-mer peptide denoted herein C3a29, and an 11-mer peptide denoted herein C3a35, where the carboxy terminus is a carboxy terminal amide, of the sequences:

C3a14:

(SEQ ID NO:12)

Asp-Val-Val-Asn-Tyr-Ile-Thr-Arg-NH2;
C3a29:

(SEQ ID NO:13)

Cys-Asp-Ser-Ser-Asn-Tyr-Ile-Thr-Arg-NH2;
C3a35:

(SEQ ID NO:14)

Ser-Asp-Ser-Ser-Asn-Tyr-Ile-Thr-Glu-Cys-Arg-NH2.

The present invention encompasses salts of the peptides, fragments, analogs, and chemical derivatives of the invention. As used herein the term “salt” refers to both salts of carboxyl groups and to acid addition salts of amino groups of the peptide molecule. Salts of carboxyl groups can be formed by means known in the art and include inorganic salts, for example aluminum, ammonium, calcium, copper, ferric, ferrous, lithium, magnesium, manganic, manganous, potassium, sodium, zinc, and the like. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Acid addition salts include, for example, salts with mineral acids such as, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like.

The term “peptide” as used herein is meant to encompass natural, non-natural and/or chemically modified amino acid residues connected one to the other by peptide or non-peptide bonds. The amino acid residues are represented throughout the specification and claims by either one or three-letter codes, as is commonly known in the art. The compounds of the invention include linear and cyclic peptides and derivatives and analogs thereof.

A “chemical derivative” as used herein refers to peptides containing additional chemical moieties not normally a part of the peptide molecule such as esters and amides of free carboxy groups, acyl and alkyl derivatives of free amino groups, esters and ethers of free hydroxy groups. Such modifications may be introduced into the peptide by reacting targeted amino acid residues of the peptide with an organic derivatizing agent that is capable of reacting with selected side chains or terminal residues.

Peptide analogs include amino acid substitutions and/or additions with natural or non-natural amino acid residues. Peptide analogs include peptide mimetics. A peptide mimetic or “peptidomimetic” is a molecule that mimics the biological activity of a peptide but is not completely peptidic in nature. Whether completely or partially non-peptide, peptidomimetics according to this invention provide a spatial arrangement of chemical moieties that closely resembles the three-dimensional arrangement of groups in the peptide on which the peptidomimetic is based. As a result of this similar active-site structure, the peptidomimetic has effects on biological systems, which are similar to the biological activity of the peptide.

The salts, analogs and the chemical derivatives of the peptides are preferably used to modify the pharmaceutical properties of the peptides insofar as stability, solubility, etc. are concerned.

### Pharmaceutical Compositions

The invention further includes pharmaceutical compositions comprising a peptide of the invention, an analog, chemical derivative, or a pharmaceutically acceptable salt thereof, together with a pharmaceutically acceptable carrier.

Without wishing to be bound to any theory, the FcεRI-β subunit can modulate or regulate the signaling or activation of the FcεRI as well as the FcγR (see, for example, Dombrowicz et al., Immunity 8:517-529, 1998). Therefore, allergic disorders caused by secretory responses of mast cells and/or basophils, which are mediated by a FcR subtype and modulated by the FcεRI-β subunit as known in the art, are encompassed in the present invention.

Apart from other considerations, the fact that the novel active ingredients of the invention are peptides, peptide analogs, peptide derivatives, or salts thereof, dictates that the formulation be suitable for delivery of these types of compounds. In general, peptides are less suitable for oral administration due to susceptibility to digestion by gastric acids or intestinal enzymes, but it is now disclosed that the compositions according to the present invention can be administered orally. The pharmaceutical composition of the present invention can be administered by any suitable means, such as topically, intranasally, subcutaneously, intramuscularly, intravenously, intra-arterially, intraarticularly, intralesionally or parenterally. Administration by inhalation is encompassed in the scope of the present invention.

The peptides of the present invention as active ingredients are dissolved, dispersed or admixed in a diluent or excipient that is pharmaceutically acceptable and compatible with the active ingredient as is well known. Suitable carriers or excipients are, for example, water, saline, phosphate buffered saline (PBS), dextrose, glycerol, ethanol, or the like and combinations thereof. Other suitable carriers are well known to those in the art. In addition, if desired, the composition can contain minor amounts of auxiliary substances such as wetting or emulsifying agents, pH buffering agents, stabilizers, binders (e.g., povidone, gelatin, hydroxypropylmethyl cellulose), lubricants, disintegrants (e.g., sodium starch glycollate, cross-linked povidone, cross-linked sodium carboxymethyl cellulose), surface active agents, thickeners, anti-oxidants, and the like.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes.

Pharmaceutical compositions for use in accordance with the present invention can be formulated in conventional manner using one or more physiologically acceptable carriers or excipients comprising auxiliaries, which facilitate processing of the active ingredients into preparations which, can be used pharmaceutically. Proper formulation is dependent upon the route of administration chosen.

For administration by inhalation, the pharmaceutical compositions according to the present invention can be delivered in the form of an aerosol spray presentation from a pressurized pack or a nebulizer with or without the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator can be formulated containing a powder mix of the peptide and a suitable powder base such as lactose or starch.

Pharmaceutical compositions, which can be used orally, include push-fit capsules made of gelatin as well as soft, sealed capsules made of gelatin and a plasticizer such as glycerol or sorbitol. The push-fit capsules can contain the active ingredients in admixture with filler such as lactose, binders such as starches, lubricants such as talc or magnesium stearate and, optionally, stabilizers. In soft capsules, the active compounds can be dissolved or suspended in suitable liquids such as fatty oils, liquid paraffin, or liquid polyethylene glycols. In addition, stabilizers may be added. All formulations for oral administration should be in dosages suitable for the chosen route of administration. For buccal administration, the compositions may take the form of tablets or lozenges formulated in conventional manner.

Dragee cores are provided with suitable coatings. For this purpose, concentrated sugar solutions can be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

For injection, the compounds of the invention can be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Penetrants for example, polyethylene glycol, are generally known in the art.

Pharmaceutical compositions for parenteral administration include aqueous solutions of the active ingredients in water-soluble form. Additionally, suspensions of the active compounds can be prepared as appropriate oily injection suspensions. Suitable natural or synthetic carriers are well known in the art (Pillai et al., Curr. Opin. Chem. Biol. 5, 447, 2001). Optionally, the suspension can also contain suitable stabilizers or agents, which increase the solubility of the active ingredients, to allow for the preparation of highly concentrated solutions. Alternatively, the active ingredient can be in a powder form for reconstitution with a suitable vehicle, e.g., sterile, pyrogen-free water, before use.

The pharmaceutical compositions of the present invention can also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a “therapeutically effective amount” means an amount of a compound effective to prevent, delay, alleviate or ameliorate symptoms of an allergic disease of the subject being treated. Determination of a therapeutically effective amount is well within the capability of those skilled in the art.

Toxicity and therapeutic efficacy of the peptides and analogs, derivatives, or salts thereof described herein can be determined by standard pharmaceutical procedures in cell cultures or in experimental animals, e.g., by determining the IC50 (the concentration which provides 50% inhibition) for a subject peptide. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition (e.g. Fingl, et al., 1975, in “The Pharmacological Basis of Therapeutics”, Ch. 1 p. 1).

Depending on the severity of the condition to be treated, dosing can also be a single administration of a slow release composition, with course of treatment lasting from several days to several weeks or until cure is effected or diminution of the disease state is achieved. The amount of a composition to be administered will, of course, be dependent on the immune status and health of the subject being treated, the severity of the disease or condition, the manner of administration, and other relevant factors.

The formulations and administration methods are intended to be illustrative and not limiting. It will be appreciated that, using the teaching provided herein, other suitable formulations and modes of administration can be readily devised.

According to some embodiments of the invention, the therapeutically effective amount of the C3a derived peptide or analog is a dosage in a range from about 0.02 mg/kg to about 10 mg/kg. Preferably, the dosage of the peptide, derivative or analog according to the present invention is in a range from about 0.05 mg/kg to about 2 mg/kg, more preferably, the dosage of the peptide, derivative or analog is in a range from about 0.1 mg/kg to about 1 mg/kg. It will be understood that the dosage can be an escalating dosage so that low dosage may be administered first, and subsequently higher dosages may be administered until an appropriate response is achieved. Also, the dosage of the composition can be administered to the subject in multiple administrations in the course of the treatment period in which a portion of the dosage is administered at each administration.

In some embodiments the peptides and derivatives and analogs thereof of the present invention are delivered to cells as modified peptides. In one embodiment the peptides of the invention are linked to a cell penetrating peptide (CPP). In one preferred embodiment the CPP is an amino acid sequence comprising the Drosophila antennapedia (ANTP) domain or a fragment thereof.

### Therapeutic Use

The peptides of the invention, as well as analogs, chemical derivatives and salts thereof can be used in the manufacture of a pharmaceutical composition or medicament for the prophylactic or therapeutic treatment of an allergic disease in mammals.

The invention relates to a method for the prevention and/or treatment of an allergic disorder mediated by a cell type selected from the group consisting of mucosal-type mast cells, serosal-type mast cells and/or basophils, without inducing an anaphylatoxic effect, said method comprising administering to a subject in need thereof a therapeutically effective amount of a pharmaceutical composition comprising as an active agent a peptide selected from the group consisting of SEQ ID NOs:3 to SEQ ID NO:8. In some embodiments, the disorder is an allergic disorder resulting from an IgE- or IgG-mediated (Type I or Type III) hypersensitivity and/or FcεRI- or FcγR-induced secretory response.

Examples of allergic diseases that can be treated by the pharmaceutical compositions of the invention include, but are not limited to, allergic rhinitis, including seasonal rhinitis and sinusitis; pulmonary diseases, such as bronchial asthma; allergic dermatosis, such as urticaria, angioedema, eczema, atopic dermatitis, and contact dermatitis; allergic conjunctivitis; gastrointestinal allergies such as those caused by food or drugs; cramping; nausea; vomiting; diarrhea; irritable bowel disease; and ophthalmic allergies such as uveitis; cheilitis; vulvitis; and anaphylaxis. The present invention is also useful in alleviating or treating the symptoms induced by exposure to toxins, including bee toxins and the like. In a certain embodiment, the allergic disorder is asthma.

Peptides having the amino acid sequence set forth in SEQ ID NO:15 to SEQ ID NO:31 have been disclosed by the applicants of the present invention in U.S. Pat. No. 6,682,740 (the content of which is incorporated by reference as if fully set forth herein) as useful for inhibiting IgE mediated (Type I) hypersensitivity where mucosal mast cells are involved. These peptides are now shown to be effective in inhibiting IgE- or IgG- and/or FcεRI- or FcγR-induced secretory responses of serosal-type mast cells and basophils.

Accordingly, the present invention further relates to a method of treating an allergic disorder mediated by a cell type selected from the group consisting of a serosal-type mast cell and a basophil comprising administering to a subject in need thereof a pharmaceutical composition comprising a therapeutically effective amount of at least one peptide selected from the group consisting of SEQ ID NOs:15 to SEQ ID NO:31.

In one embodiment, the peptide to be used in a method for treating an allergic disorder where serosal mast cells and/or basophils are involved is the peptide herein denoted C3a7 set forth in SEQ ID NO:25, a 9-mer peptide derived from the 56-64 sequence of human complement peptide C3a, of the sequence:

Cys-Cys-Asn-Tyr-Ile-Thr-Glu-Leu-Arg

or an analog, derivative or salt thereof.

In yet a further embodiment, the peptide of the invention to be used in a method for treating an allergic disorder where serosal-type mast cells and/or basophils are involved is the peptide herein denoted C3a9 set forth in SEQ ID NO:26, an 8-mer peptide derived from the 55-62 sequence of the human complement peptide C3a, of the sequence:

Asp-Cys-Cys-Asn-Tyr-Ile-Thr-Arg

or an analog, derivative or salt thereof.

In another embodiment, the peptide of the invention to be used in a method for treating an allergic disease where serosal-type mast cells and/or basophils are involved is the peptide herein denoted C3a11 set forth in SEQ ID NO:27, a 7-mer analog derived from the 55-61 sequence of the human complement peptide C3a, of the sequence:

Asp-Ser-Ser-Asn-Tyr-Ile-Arg

or an analog, derivative or salt thereof.

In still further embodiments, the peptides of the invention to be used in a method for treating an allergic disease where serosal-type mast cells and/or basophils are involved are the 14-mer C3a4, 20-mer C3a5, 15-mer C3a6, 10-mer C3a10, of the sequences:

C3a4:

(SEQ ID NO:28)

Lys-Val-Phe-Leu-Asp-Cys-Cys-Asn-Tyr-Ile-Thr-Glu-
Leu-Arg;
C3a5:

(SEQ ID NO:29)

Lys-Lys-Val-Phe-Leu-Asp-Cys-Cys-Asn-Tyr-Ile-Thr-
Glu-Leu-Arg-Arg-Gln-His-Ala-Arg;
C3a6:

(SEQ ID NO:30)

Lys-Val-Phe-Leu-Asp-Ala-Ala-Asn-Tyr-Ile-Thr-Glu-
Leu-Arg-Arg;
C3a10:

(SEQ ID NO:31)

Arg-Arg-Cys-Cys-Asn-Tyr-Ile-Thr-Arg-Arg;

or the analogs, derivatives, or salts thereof.

In some preferred embodiments, the carboxy terminus of the peptides set forth in any one of SEQ ID NOs:25 to SEQ ID NO:31 is a carboxy terminal amide.

For the treatment of hay fever, for example, pharmaceutical compositions in the form of spray or aerosol can be appropriate for administration to subjects in need to prevent the development of allergy in the pollen-season. Moreover, it is well known that the bronchial mucosal surface is the first contact site for inhaled allergens and, consequently, the response of mast cells to the inhibitory peptides of the invention administered as spray may be very effective.

Allergic disorders associated with serosal mast cell activation include, but are not limited to, Type I or Type III immediate hypersensitivity reactions such as gastrointestinal allergies, cramping, nausea, vomiting, and diarrhea.

The peptides of the present invention can be administered as pharmaceutical compositions as a monotherapy, or in combination with other therapeutic agents, such as, for example, other anti-inflammatory agents. Combination therapies can involve the administration of the pharmaceuticals as a single dosage form or as multiple dosage forms administered at the same time or at different times.

The invention will now be illustrated by the following non-limiting examples.

### EXAMPLES

### Material and Methods

Reagents and cell culture media. Tissue culture media and supplements were purchased from Invitrogen Life Technologies or from Gibco (Grand Island, N.Y.). Triton X-100, p-nitrophenyl-N-acetyl-β-D-glucosamine and anti-phosphotyrosine Ab PT-66 were from Sigma (Sigma-Aldrich Kft., Hungary). 2,4-dinitrobenzene sulphonic acid-conjugated bovine serum albumin (DNP11-BSA), DNP-coated beads and murine DNP-specific monoclonal A2 IgE were kindly donated by Mr. Arieh Licht (Rehovot, Israel). Horseradish peroxidase (HRPO)-conjugated anti-mouse IgG and HRP-labeled anti-rabbit IgG were purchased from Sigma-Aldrich, and anti-Lyn Ab was from BD Transduction Laboratories. Enhanced chemiluminescence reagent (ECL) was purchased from Amersham Biosciences (UK), and materials used for SDS-gel electrophoresis were obtained from Bio-Rad (CA, USA). The Fluo-3 AM dye was obtained from Calbiochem. C3a and C5a were isolated as described (see Erdei el a., Int. Immunol. 7: 1433-1439, 1995).

Synthetic peptides and protein labeling. Peptide synthesis was carried out by solid phase technique utilizing ‘Boc chemistry’ (Merifield et al. Biochem. 14: 1385-1390, 1964) on MBHA-resin. Peptides were purified and characterized by reversed phase HPLC and mass spectrometry. Peptide C3a9 was labeled with Cy5 and A2IgE with Cy3 as given by the instructions (labeling protocol for 0.1 M NaHCO3) provided by Amersham-Pharmacia (NJ, USA).

Cells. Bone marrow derived mast cells (BMMC) were prepared from Balb/c mice as described by Nagao et al. (Science 212: 333-335, 1981). After 3 weeks, an approximately 95% pure mast cell population was obtained, showing high expression of FcεRI and stem cell factor receptor (c-kit), as measured by flow cytometry.

RBL-2H3 cell line, obtained from Dr. Reuben Siraganian, NIH, Bethesda Md., was maintained in Dulbecco's Modified Essential Medium (DMEM) supplemented by 5% FCS, 2 mM glutamine and antibiotics in a humidified atmosphere with 5% CO2 at 37° C. For the experiments, cells were harvested following detachment by 15 min incubation with 10 mM EDTA in DMEM.

Rat peritoneal mast cells (RPMC) were isolated as previously described (Kim et al. J. Immunol. 162:4960-4965, 1981). Mast cell preparations were ca. 95% pure, as evaluated by flow cytometric monitoring of FcεRI surface expression. Compound 48/80, a specific activator of serosal type mast cells elicited about 50% release of the total β-hexosaminidase content of the isolated rat peritoneal mast cells.

Secretory response of mast cells. Mediator secretion by mast cells in response to stimulation by FcεRI clustering was monitored by measuring activity of the secreted granular enzyme β-hexoseaminidase as described (see Erdei et al. Int. Immunol. 7: 1433-1439, 1995). To study the effect of C3a and its derivatives on antigen-induced response, mast cells sensitized with saturating concentrations of DNP-specific A2 IgE were preincubated with a concentration range of the various peptides for 5 min at room temperature before exposure to suboptimal antigen concentrations (5 ng/ml).

Measurement of TNF-α by ELISA. TNF-α secretion by RBL-2H3 in response to FcεRI clustering in the absence and presence of the peptides was determined with a rat TNF-α ELISA kit (R&D systems, UK).

Immunoprecipitation and Western blot. Mast cells were seeded in 10 cm Petri dishes (7×106 cells/dish) and saturated with A2IgE at 37° C. in DMEM overnight. After washing they were treated with peptides for 5 min and FcεRI was clustered by incubating with 5 ng/ml DNP11-BSA at 37° C. for 2 min. The reaction was terminated by 500 μl lysis buffer (1% Triton X-100, 50 mM HEPES, 100 mM NaF, 10 mM EDTA, 2 mM sodium orthovanadate, 10% glycerol, 10 mM sodium pyrophosphate, protease inhibitor cocktail 1:200, pH 7.4) per dish. The cells were scraped and the protein content of the post nuclear supernatants was adjusted to equal values using the Bradford-assay prior to precipitation by PT-66 phosphotyrosine specific antibody coated beads (10 μl beads per sample). The proteins were eluted by sample buffer containing 2-mercaptoethanol and were separated by SDS-PAGE, electrotransferred onto a nitrocellulose membrane and developed using the indicated antibodies and detected by ECL.

Monitoring free cytosolic Ca2+-ion concentrations. A total of 5×106 IgE-sensitized cells in 0.5 ml RPMI-1640 medium were loaded with 5 μM Fluo-3/AM indicator and 30 μg/ml Pluronic F-127 for 30 min at 37° C. After washing, the cells (at 5×105 cells/ml) were incubated with C3a7 or C3a9 peptides at 200 μM for 5 min at 37° C. Twenty seconds after initiation of flow cytometric recording, 5 ng/ml of antigen (DNP11-BSA) was added to the cells and the changes in free calcium ion concentration was followed in the time-resolved mode of a Becton-Dickinson FACSCalibur flow cytometer. Data acquisition and analysis were performed with the CELLQuest software (Becton-Dickinson, Franklin Lakes, N.J., USA).

Laser Scanning Confocal Microscopy. RBL-2H3 cells were harvested and incubated either with 5 μM of the Cy3-conjugated IgE or simultaneously also with 200 μM Cy5-conjugated C3a9 peptide for 25 min at 4° C. After washing, the cells were fixed with 2% paraformaldehyde on ice for 20 min and then mounted on a coverslip precoated with 0.1% poly-L-lysine. The fluorescence signals from the Cy3-labeled IgE and the Cy5-peptide were analyzed in the green (excitation by 543 nm He—Ne laser) and red (excitation by 632 nm He—Ne laser) optical channels of a Zeiss LSM5 laser scanning confocal microscope. The cells were optically sliced to 512×512 pixel sections with 0.5 μm thickness. Estimates of the cross-correlation coefficients between fluorescence intensities as a measure of co-localization was carried out as described earlier (Vereb et al. Proc. Natl. Acad. Sci. USA 97: 6013-6018, 2000.(

Flow cytometric Fluorescence Resonance Energy Transfer (FRET) measurements. FRET between FITC-labeled C3a9 peptide (donor) and Cy3-labeled IgE (acceptor) both bound to the surface of RBL-2H3 cells was measured and evaluated using Becton Dickinson FACSStar Plus flow cytometer. RBL-2H3 cells were labeled with saturating concentrations of Cy3-IgE while the FITC-C3a9 peptide was at 150 μM, i.e., at doses similar to those employed in the functional studies.

Covalent cross linking of C3a to BMMC. BMMC were washed and incubated with C3a (50 μg/ml per 15−20×106 cells/sample, in PBS) for 10 min at room temperature. After another incubation of 20 min at room temperature with 5 mM of the cross linking reagent bis-(sulphosuccinimidyl)-suberate (BS3, PIERCE, Ill., USA) cells were washed and lysed. Polyclonal rabbit antibodies specific to C3a (Behringwerke AG, Germany) were used for immunoprecipitation. Western blotting was carried out by an FcεRI β chain specific antibody kindly provided by Jean Pierre Kinet (Harvard University, Boston, USA). As secondary antibody, HRPO-conjugated goat anti-mouse IgG (DAKO, Frank Diagnosztika Kft, Hungary) was used. Detection was performed by ECL.

Surface Plasmon Resonance (SPR) measurements. SPR measurements were performed using a BIACORE instrument Model 2000 (Pharmacia, Sweden). Peptides with the following sequences were synthesized and employed: (1) the 1st extracellular loop of the rat and human FcεRI β chain: STLQTSDFDDEVLLLYRAGYPF (SEQ ID NO:39) and SVLDISHIEGDIFSSFKAGY (SEQ ID NO:40); and (2) the 2nd extracellular loop of the rat and human FcεRI β chain: NNSAYMNYCKDITEDDGCFVTS (SEQ ID NO:41) and KSLAYIHIHSCQKFFETKCFMAS (SEQ ID NO:42), respectively.

All peptides were biotinylated at their N-terminal amino group and bound to the streptavidin coated sensor chips (BIACORE, Sweden) at low densities, ranging between 30 and 60 resonance units (RU). C3a and C5a solutions in distilled water at 5 different concentrations, ranging from 10.5 mM to 656 nM, were injected at a flow rate of 20 μl/min. The surface of the chips was regenerated between measurements by 0.1 M HCl. Data was analyzed with BIAEVALUATION 3.0 software. The observed association rate constants (kobs) were plotted as the function of the C3a concentration and the slope of each plot was taken as specific rate constant of association (kon), while the y intercept was taken as the dissociation rate constant (koff), then Kd was calculated as koff/kon.

Passive Systemic Anaphylaxis. Mice (Balb/c) were anesthetized with 300-400 μl of avertin and injected with 3 μg of the IgE class DNP-specific monoclonal antibody (SPE-7, Sigma) in 200 μl PBS by retroorbital injection (or tail vein). After 24 hours, mice were anesthetized with 300-400 μl of avertin and exposed to ca. 100 μl of the indicated peptide (inhibitory, SEQ ID NO:11, a control reversed peptide of SEQ ID NO:35 or peptide GAKDGNEYI-COOH of SEQ ID NO:36) solution in PBS positioned onto the nostrils. This was followed by injection of the antigen (DNP-derivatized human serum albumin, HSA, 100 μg in 200 μl of PBS) or PBS only, by retroorbital injection (or tail vein). After 1.5 minutes, mice were sacrificed by cervical dislocation and cardiac puncture performed. Heparin-rinsed syringes were now used for aspiration. Blood samples were spun at 4 degrees for 10 minutes at 8000 rpm in tabletop. Histamine was assayed by the Immunotech protocol for competitive ALP assay.

Inhibitory effect of the peptides on pulmonary functions. Mice (Balb/c, female, 8 weeks old) were first sensitized by ip injection of the antigen (Ag) ovalbumin. Thereafter, the mice were challenged 4 times by aerosol inhalation of the same Ag dispersed by nebulizer in a plexiglass chamber for about 20 min at one-week intervals. On day 28, the mice were first treated with an aerosol of the tested peptides dissolved in 0.1 M NaHCO₃ and dispersed as above. As a result, the aerosol was inhaled by the animals for about 10 to 20 minutes. After inhalation, the animals were immediately challenged by inhalation (as above) of the sensitizing antigen (5% OVA in PBS) for 20 min. At the end of this treatment, the mice were immediately tested for pulmonary functions in conscious, freely moving state using plethysmography.

The degree of bronchial constriction was monitored by the enhanced pause and its relation to airway resistance, impedance and intrapleural pressure in the mouse. Bronchoalveolar lavage (BAL) samples were obtained from these mice by cannulating the trachea, injecting 0.8 ml ice-cold saline (×2) and subsequently aspirating the BAL fluid. Following these tests, mice were sacrificed using general anesthesia with brevital (1 mg/ml). Their chests wall were opened, blood withdrawn and lungs were perfused with cold PBS and examined for cytology and histology.

### Example 1

### Peptides Derived from Human C3a

Table 1, set forth hereinbelow, provides a list of the synthesized peptides, their amino acid sequences, mass spectrometry data and name codes. Peptides C3a1, C3a3, C3a55, and GAK peptide (SEQ ID NOs:33 to SEQ ID NO:36) were used as control peptides. The peptides listed in Table 1 were synthesized using the ‘Boc chemistry’ as disclosed herein above. In addition, all the peptides were further prepared as amidated peptides. It is to be noted that the method of peptide synthesis is not intended to be limiting.

| | SEQ | Mass Spectra | | Name |
|---|---|---|---|---|
| Peptide Sequence | ID | calc. | actual | Code |
| dAdADSSNYITR | 7 | 1097 | 1096 | C3a32 |
| SDSSNYITR | 11 | 1042 | 1041 | C3a31 |
| DVVNYITR | 12 | 979 | 978 | C3a14 |
| CDSSNYITR | 13 | 1076.1 | | C3a29 |
| SDSSNYITECR | 14 | 1284 | | C3a35 |
| CCNYITELR | 25 | 1113.3 | 1113.8 | C3a7 |
| DCCNYITR | 26 | 986.14 | 986.2 | C3a9 |
| DSSNYIR | 27 | 852.90 | 853.6 | C3a11 |
| KVFLDCCNYITELR | 28 | 1716.05 | 1716.5 | C3a4 |
| KKVFLDCCNYITELRRQHAR | 29 | 2492.9 | 2493.0 | C3a5 |
| KVFLDAANYITELRR | 30 | 1808.1 | 1808.8 | C3a6 |
| RRCCNYITRR | 31 | 1339.61 | 1340.1 | C3a10 |
| DSSNYITR | 32 | 955 | | C3a14 |
| SVQLTEKRMDKVGKYPKELR | 33 | 2404.88 | 2406.5 | C3a1 |
| RQHARASHLGLAR | 34 | 1471.70 | 1474.4 | C3a3 |
| RTIYNSSDS | 35 | 1042 | 1041 | C3a55 |
| GAKDGNEYI | 36 | | | GAK |
| CCNYG | 37 | 558.64 | | C3a13 |
| DVSNYITR | 38 | | | |

### Example 2

### The Peptides' Inhibitory Capacity on the Secretory Response of Mucosal Type Mast Cells

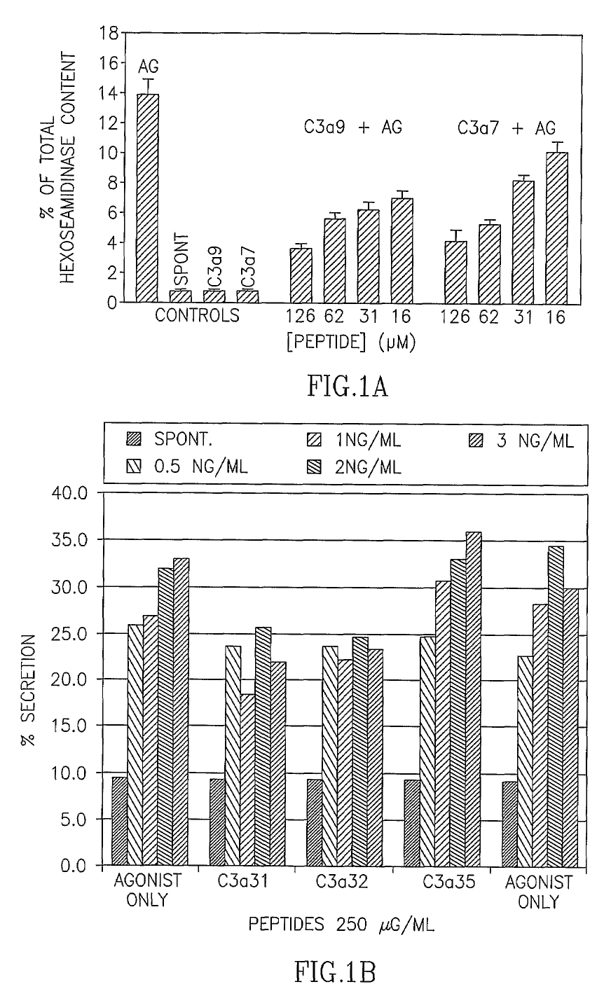

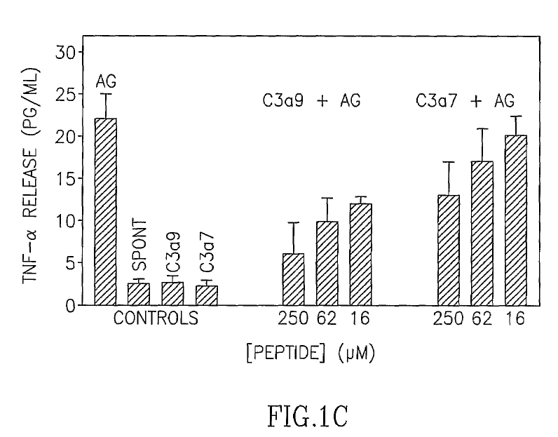

In earlier experiments the inventors of the present application have identified the C3a sequence motif responsible for inhibiting the IgE-mediated stimulation of RBL-2H3 cells (Erdei et al., Immunol. Lett. 68: 79-82, 1999). The results have clearly demonstrated that the C-terminal sequence of C3a (residues 65-77)—known to be of major importance in exerting anaphylatoxic and chemotactic activity of the complement-peptide—is not involved in that inhibition. However, upstream sequences, comprising residues 56-64 (CCNYITELR, designated C3a7) are involved. Several analogs of this sequence were now synthesized, out of which an octapeptide: DCCNYITR, designated C3a9, is shown to be effective in inhibiting FcεRI-mediated secretion of mucosal type mast cells of the RBL-2H3 line (FIG. 1). IgE-sensitized cells were incubated with the peptides for 5 min prior to the stimulation with a suboptimal (5 ng/ml) antigen dose, followed by measuring activity of the secreted granular enzyme, β-hexosaminidase. FIG. 1A shows the dose-dependent inhibition exerted by these peptides on these cells' response.

The effect of the peptides designated C3a31, C3a32, and C3a35 on mucosal-type mast cell secretory response is shown in FIG. 1B. As shown in FIG. 1B, peptides C3a31 and C3a32 were found to inhibit IgE-mediated β-hexoseaminidase secretion from RBL-2H3. The peptides C3a11 and C3a13 were tested and shown to inhibit IgE-dependent degranulation of RBL-2H3 cells.

Similar experiments were carried out using bone-marrow derived mast cells (BMMC). C3a was found to inhibit the IgE-mediated secretory response of these mucosal type mast cells as well (Table 2), while C5a had no effect. As shown in Table 2, the inhibitory effect of the peptides C3a7 and C3a9 on BMMC's secretory response is comparable to that exerted on RBL-2H3 cells. The control peptide of the sequence DVSNYITR had no effect on either system.

TABLE 2
Inhibitory effect (IC50, μM) of C3a and C3a-derived
peptides on the IgE-mediated degranulation of RBL-2H3,
BMMC and rat peritoneal mast cells (RPMC).

| | RBL-2H3 | BMMC | RPMC |
|---|---|---|---|
| C3a* | 2.4 | 0.2 | Activation* |
| C3a7 | 52 | 69 | 123 |
| C3a9 | 51 | 69 | 105 |

| | | |
|---|---|---|
| Control | No effect | No effect |
| peptide | | |

For the degranulation assay, suboptimal antigen-dose was used.
*C3a, when added to non-sensitized cells, stimulated only the serosal type RPMC, but not the mucosal type RBL-2H3 and BMM cells.

### Example 3

### The Peptides' Inhibitory Capacity on the Secretory Response of Serosal Type Mast Cells

While mucosal type mast cells are non-responsive to peptidergic stimuli, the serosal type ones have been known for a long time to be stimulated by cationic secretagogues. The anaphylatoxic peptides C3a and C5a initiate the cells' secretory response by binding to their respective receptors expressed by serosal type mast cells. To investigate the effect of the peptides on this mast cell-type, RPMC were used. As expected, these cells did respond to both C5a and C3a (Table 2). However, degranulation was inhibited when peptides C3a7 or C3a9 were added to the IgE-sensitized RPMC and stimulated, 5 min later with a suboptimal antigen dose (Table 2).

### Example 4

### Peptides C3a7 or C3a9 Suppress the FcεRI-Clustering Induced TNF-α Secretion

The effect of the peptides on mast cells' late phase response was determined by measuring TNF-α secretion. To this end, supernatants were taken 24 hours after stimulation of RBL-2H3 cells in the absence or presence of peptides C3a7 or C3a9 and the secreted cytokine concentration was determined by ELISA. As shown in FIG. 1C, both peptides dose-dependently inhibited the release of this inflammatory cytokine. When added alone, the peptides had no effect.

### Example 5

### Tyrosine Phosphorylation of Lyn, PI-3K and the β Subunit of FcεRI is Suppressed by the Inhibitory Peptides

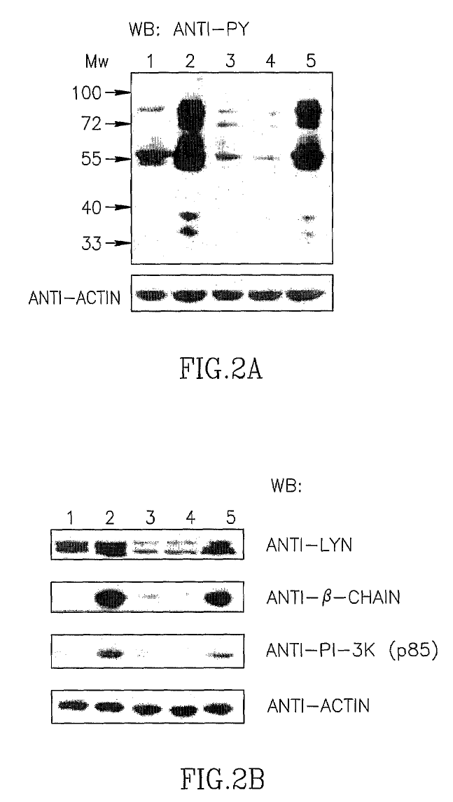

FIG. 2A shows that the FcεRI clustering induced enhancement of protein tyrosine phosphorylation of several intracellular proteins is markedly reduced upon exposure of the RBL-2H3 cells to 200 μM C3a7 (lane 3) or C3a9 (lane 4) prior to antigen stimulation. Based on this result and on earlier findings (FIG. 1), the FcεRI-proximal events were investigated. These are known to include phosphorylation by the src-family PTK Lyn of the ITAMs of the FcεRI β and γ subunits. As shown in FIG. 2B, phosphorylation of Lyn was strongly reduced when the cells were stimulated by antigen in the presence of C3a7 or C3a9, while the control peptide had no effect. Phosphorylation of the FcεRI β chain was also examined and found to be decreased compared to that of cells treated by the control peptide (FIG. 2B). Tyrosine phosphorylation of PI-3K, the enzyme regulating phosphatidyl inositides' phosphorylation levels and therefore being a key coupling element, was also found to be reduced upon exposure to peptides C3a7 and C3a9 (FIG. 2B). These peptides did not cause by themselves any change in the pattern of tyrosine phosphorylated proteins of RBL-2H3 cells.

The inhibitory action of the peptide C3a31 on the FcεRI coupling cascade was also investigated using the rat mucosal-type RBL-2H3 line. Results of these experiments have shown that peptide C3a31 inhibited the phosphorylation of the protein tyrosine kinase Lyn and of the FcεRI β-chain. The inhibition on Lyn phosphorylation was observed after 1 min, and that on PAG phosphorylation (PAG has an important role in the regulation of src family kinases) after 3 min. However, C3a31 was also shown to enhance Lyn phosphorylation after 3 min. In addition, C3a31 inhibited Dok-1 phosphorylation after 5 min, but had no effect after 8 min. It also inhibited the phosphorylation of PLCγ-2.

### Example 6

### The Peptides Inhibit the Antigen-Induced Transient Elevation of [Ca²⁺]ᵢ

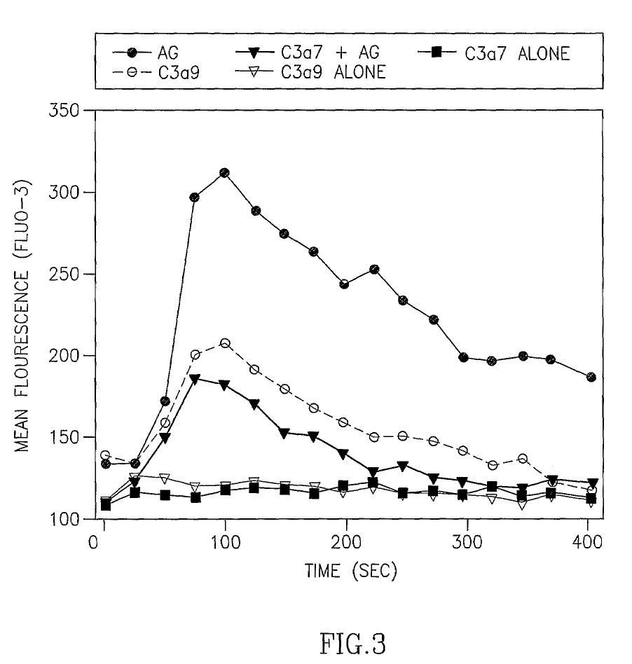

As the transient increase in [Ca²⁺]ᵢ is one of the earliest events induced in antigen activated cells, the effect of C3a7 and C3a9 on this process in RBL-2H3 cells was examined. As illustrated in FIG. 3, this process was markedly inhibited when peptides C3a7 or C3a9 were added to the cells 5 minutes before antigenic challenge. Both the initial rapid rise of [Ca²⁺]ᵢ as well as the subsequent sustained elevated level, assigned to the ion influx from the extracellular medium were suppressed. In controls, where the peptides were added alone, no effect could be resolved.

### Example 7

### C3a Binds to the FcεRI β-Chain

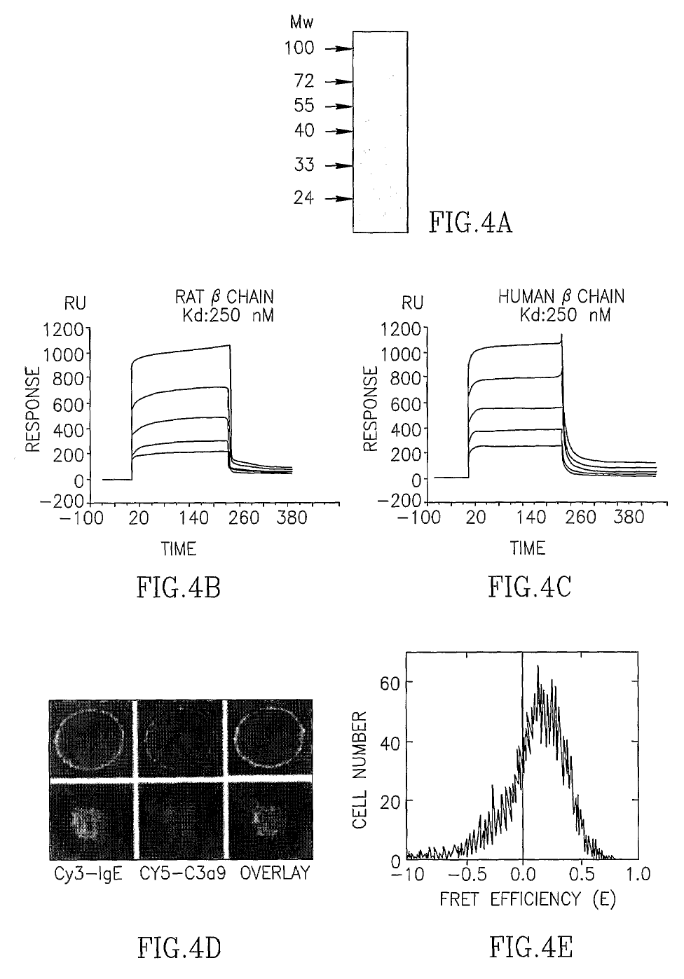

Covalent cross-linking of C3a to BMMC. Since no C3a-receptors could be detected on mucosal type mast cells, the possibility that the peptides exert their inhibitory effect via interaction with a different cell membrane component, probably the type 1 Fcε-receptor, was examined. In earlier experiments the inventors observed no interference by C3a with the interaction between IgE and the FcεRI, consequently it was assumed that it does not interact with the α-chain of the tetrameric FcεRI-complex. Since the ITAM-bearing β-chain had earlier been reported to act as an amplifier of the FcεRI response, the interaction of C3a with this membrane protein on BMMC was determined. Cells were incubated with C3a followed by adding the covalent cross linking agent, BS³-reagent. This was followed by cell lysis and immunoprecipitation with polyclonal C3a-specific antibodies. Protein samples were separated by SDS-PAGE and analyzed by Western-blotting, using FcεRI β-chain specific antibodies. As shown in FIG. 4A, only a single protein was observed (lane 1), while none could be resolved in the control sample (lane 2). Due to the specific reactions of the covalently cross linked complex to each component, i.e. for C3a on one hand and for the β-chain on the other, the only band appearing in Lane 1 with the mass of ˜45 kDa is a covalent complex of C3a (9 kDa) and the β-chain (36 kDa). These results are in full agreement with our earlier ones obtained using RBL-2H3 cells (Erdei et al., 1999, ibid).

Surface plasmon resonance (SPR) measurements of the interaction between C3a and the FcεRI β-chain. To further investigate the interaction of C3a with the β-chain of FcεRI, SPR measurements were carried out using immobilized oligo-peptides with sequences of the first and second extracellular loops of both, the human and the rat tetraspan molecules. Human C3a and, as control, C5a were used as analytes. As shown in FIGS. 4B and C, C3a bound to the first extracellular loop of both the human and rat protein with Kd values of 250 nM and 520 nM, respectively. No interaction could be detected between C3a and the second extracellular loop of the cell membrane protein. C5a did not react with any tested sequences of FcεRI β-chain.

### Example 8

### C3a9 is Co-Localized with FcεRI-Bound IgE on Intact RBL-2H3 Cell

In order to visualize details on intact cells of the spatial relation between the C3a-derived peptides and the IgE bound to the FcεRI, confocal laser scanning microscopic measurements were performed employing Cy3-conjugated IgE and Cy5-conjugated C3a9 peptide on RBL-2H3 cells. A pixel-by-pixel analysis of the cross-correlation between Cy3 and Cy5 emission signals (cross-correlation coefficient: >0.54) indicated that the cell-bound peptide is highly co-localized with the IgE bound to the FcεRI α subunit. This is in agreement with all the other findings and strongly supports that the peptide's interaction site is within the multisubunit FcεRI receptor complex of mast cells (FIG. 4D). In addition, human monocytes lacking the β-chain had also been tested by confocal fluorescence microscopy and no detectable peptide-binding was found.

The above results were further supported by results of FRET measurements indicating proximity at the nanometer scale. A flow cytometric resonance energy transfer (FCET) efficiency histogram (FIG. 4E), showing a mean FRET efficiency of 24% clearly indicated a molecular proximity between FITC-C3a9 peptide and the Cy3-IgE bound to the surface of RBL-2H3 cells.

### Example 9

### Inhibition of Passive Systemic Anaphylaxis

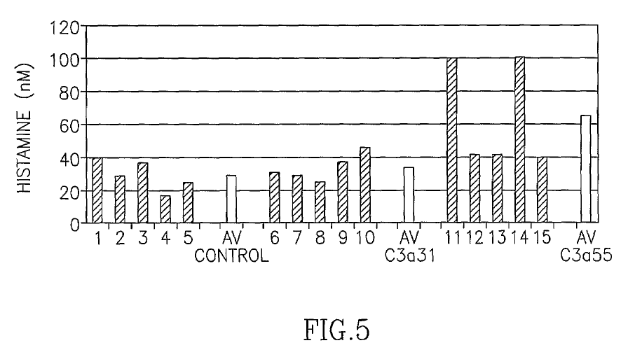

The effect of C3a derived peptides on passive systemic anaphylaxis in mice was examined. As shown in FIG. 5, C3a31 amide was found to reduce the symptoms of passive systemic anaphylaxis as monitored by assaying histamine levels in mice blood. The reverse peptide denoted C3a55 amide was ineffective (FIG. 5). A control group was treated with a non-relevant peptide having the amino acid sequence GAKDGNEYI-COOH of SEQ ID NO:36 prior to the injection of the antigen DNP-HSA.

### Example 10

### Inhibitory Capacity of C3a Derived Peptides in an Asthma Model

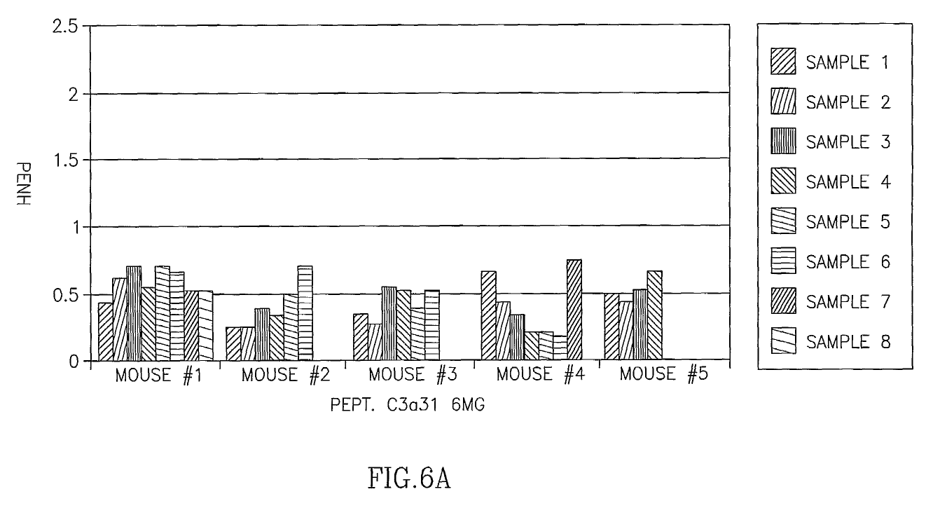

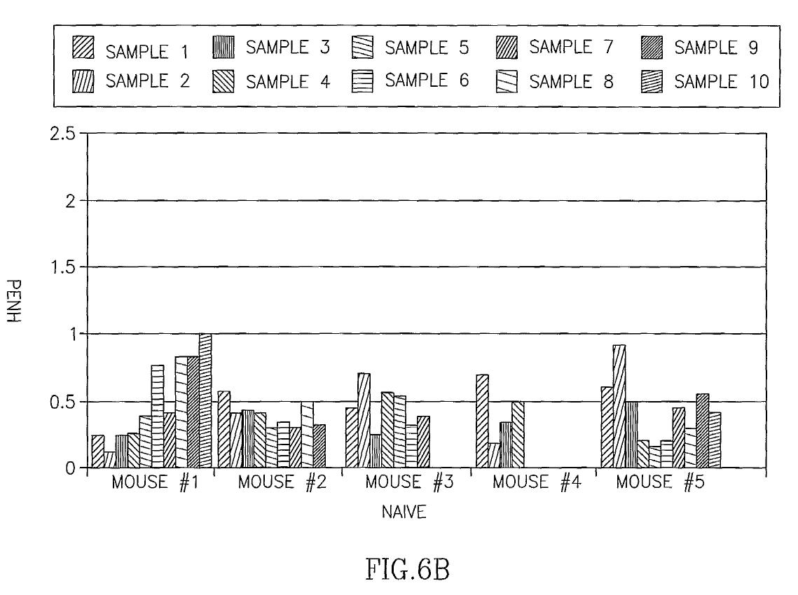

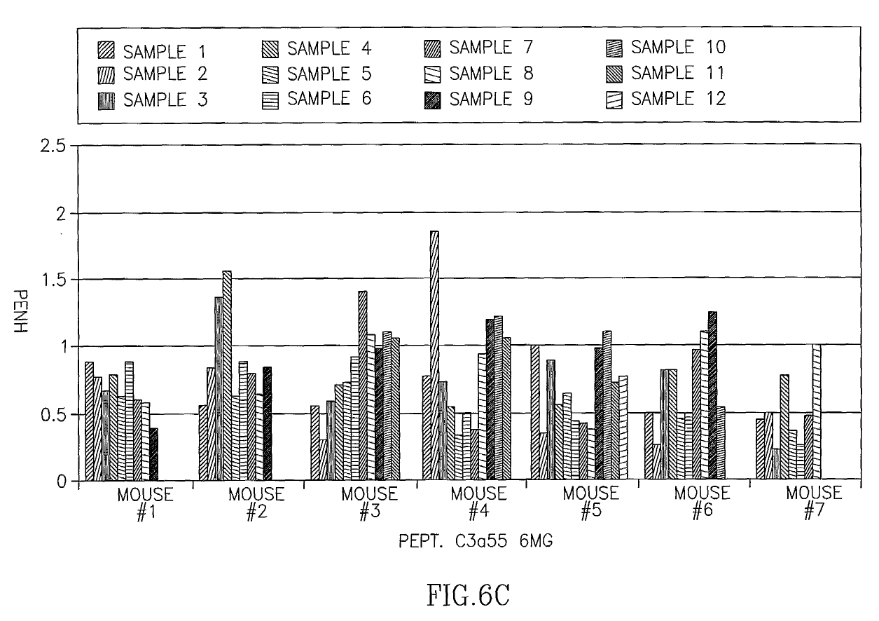

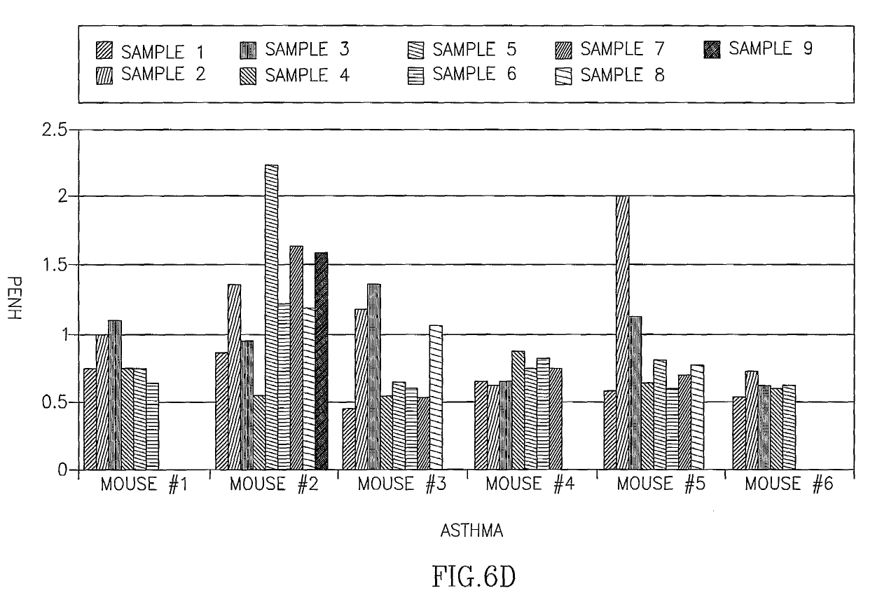

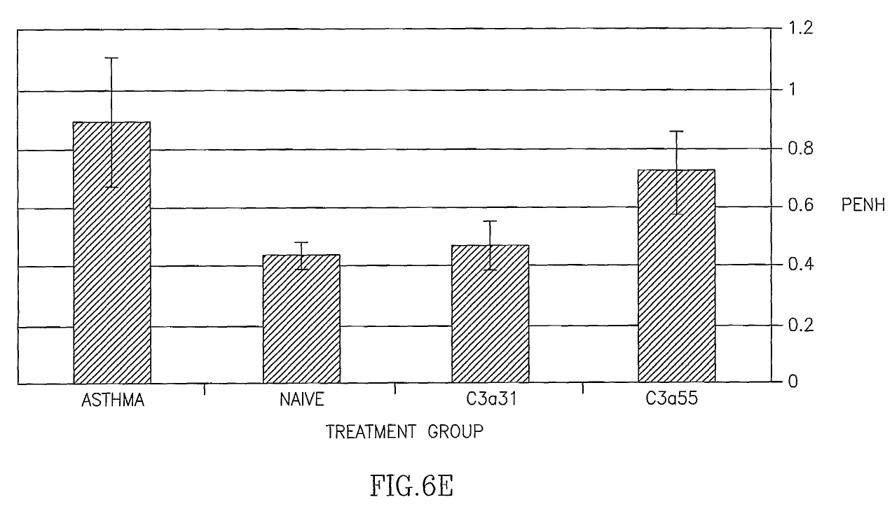

Lung function was assayed as described in the Experimental section in mice exposed (or not, i.e. naïve) to sensitization by an initial antigen (ovalbumin) injection that was followed by 4 challenges by exposure to this antigen's aerosol. Animals were finally exposed to an aerosol of either buffer alone, to buffer containing peptide C3a31 or to buffer containing a reverse peptide denoted C3a55, prior to the final (usually fifth) challenge by antigen. FIGS. 6A-D illustrate the protective capacity of peptide C3a31. FIG. 6A illustrates the individual values of lung airway resistance in naïve mice; FIG. 6B illustrates the individual values of lung airway resistance in asthmatic mice; FIG. 6C illustrates the individual values of lung airway resistance in “asthmatic” mice treated prior to Ag (ovalbumin) aerosol challenge by peptide C3a31 and FIG. 6D illustrates the individual values of lung airway resistance in “asthmatic” mice treated prior to Ag challenge with peptide C3a55. FIG. 6E shows the average values and standard deviation of the results shown in FIGS. 6A-D. These results unequivocally show that the C3a31 peptide is effective in reducing the antigen-induced symptoms in an animal model of asthma.

Table 3 provides results of analysis of the cells present in bronchi alveolar lavage (BAL) of mice treated by the same protocols as those subjected to analysis of lung function. As shown in the Table 3, treatment with peptide C3a31 markedly affected the type of cells detected in the BAL reducing primarily the number of neutrophils.

TABLE 3
Impact of exposure to peptide's aerosol on cell
distribution in broncho alveolar lavage (BAL).

| | Naive | OVA | OVA + Pep (1) |
|---|---|---|---|
| Neutrophils | 1.7% | 19% | 13% |
| Eosinophils | 0 | 25% | 4% |
| Lymphocutes | 1.3% | 8% | 81% |
| Macrophages | 97% | 47% | 1% |
| Mast Cells | 0 | 0 | 4% |

The present results have clearly established the marked capacity of the C3 a derived peptides to inhibit allergy related symptoms. This was observed in cell cultures examining both mucosal-type and serosal-type mast cells as well as in animal in vivo models showing a similar marked suppression of the asthma like symptoms.

It will be appreciated by persons skilled in the art that the present invention is not limited by what has been particularly shown and described herein above. Rather the scope of the invention is defined by the claims that follow.

1. A peptide derived from the sequence of amino acids 55-64 (SEQ ID NO:2) of human complement C3a having an amino acid sequence of general formula I:
X1-Asp-X2-Asn-Tyr-Ile-Thr-X3 (SEQ ID NOs:3 to 10)
wherein
X1 is selected from the group consisting of hydrogen, lower alkanoyl, Cys, Ser, D-Ala, and D-Ala-D-Ala;
X2 is selected from the group consisting of Ser-Ser and Val-Val; and
X3 is selected from the group consisting of Arg, Arg-NH2, Glu-Cys-Arg, and Glu-Cys-Arg-NH2;
or an analog, chemical derivative, or a pharmaceutically acceptable salt thereof; with the proviso that when X1 is hydrogen or lower alkanoyl and X3 is Arg or Arg-NH2, then X2 is Val-Val.

2. The peptide according to claim 1, wherein the secretory response is induced by a stimulus selected from the group consisting of (i) IgE- or IgG-mediated triggering, and (ii) FcεRI or FcγR clustering.

3. The peptide according to claim 1 having an amino acid sequence selected from the group consisting of:
(SEQ ID NO:7)
(a) D-Ala-D-Ala-Asp-Ser-Ser-Asn-Tyr-Ile-Thr-Arg-Z;
(SEQ ID NO:11)
(b) Ser-Asp-Ser-Ser-Asn-Tyr-Ile-Thr-Arg-Z;
(SEQ ID NO:12)
(c) Asp-Val-Val-Asn-Tyr-Ile-Thr-Arg-Z;
(SEQ ID NO:13)
(d) Cys-Asp-Ser-Ser-Asn-Tyr-Ile-Thr-Arg-Z;
(SEQ ID NO:14)
(e) Ser-Asp-Ser-Ser-Asn-Tyr-Ile-Thr-Glu-Cys-Arg-Z;
(f) an analog of (a), (b), (c), (d) or (e);
(g) a chemical derivative of (a), (b), (c), (d),
(e), or (f);
and
(h) a salt of (a), (b), (c), (d), (e), (f) or (g);
where Z designates a terminal carboxy acid, amide, or alcohol.

4. The peptide according to claim 3, wherein Z is a carboxy terminal amide.

5. The peptide according to claim 4 having an amino acid sequence set forth in SEQ ID NO:7, wherein Z is a carboxy terminal amide.

6. The peptide according to claim 4 having an amino acid sequence set forth in SEQ ID NO:11, wherein Z is a carboxy terminal amide.

7. A pharmaceutical composition comprising as an active agent a peptide according to claim 1, and a pharmaceutically acceptable carrier.

8. The pharmaceutical composition according to claim 7, wherein the peptide having an amino acid sequence selected from the group consisting of SEQ ID NOs:3 to SEQ ID NO:14, and analogs and chemical derivatives thereof.

9. The pharmaceutical composition according to claim 8, wherein the peptide having an amino acid sequence set forth in SEQ ID NO:7, optionally having a carboxy terminal amide.

10. The pharmaceutical composition according to claim 8, wherein the peptide having an amino acid sequence set forth in SEQ ID NO:11, optionally having a carboxy terminal amide.

11. A method for the prevention and/or treatment of an allergic disorder comprising the step of administering to a subject in need thereof a pharmaceutical composition comprising a therapeutically effective amount of a peptide according to claim 1, and a pharmaceutically acceptable carrier, thereby preventing or treating the allergic disorder.

12. The method according to claim 11, wherein the peptide having an amino acid sequence selected from the group consisting of SEQ ID NOs:3 to SEQ ID NO:14 optionally having a carboxy terminal amide.

13. The method according to claim 12, wherein the peptide having an amino acid sequence set forth in SEQ ID NO:7 optionally having a carboxy terminal amide.

14. The method according to claim 12, wherein the peptide having an amino acid sequence set forth in SEQ ID NO:11 optionally having a carboxy terminal amide.

15. The method according to claim 11, wherein the allergic disorder resulting from IgE- or IgG-mediated (Type I or Type III) hypersensitivity and/or FcεRI or FcγR-induced secretory response.

16. The method according to claim 11, wherein the allergic disorder is mediated by a cell type selected from the group consisting of mucosal-type mast cells, serosal-type mast cells and basophils.

17. The method according to claim 16, wherein the allergic disorder is selected from the group consisting of allergic rhinitis, pulmonary diseases, allergic dermatosis, allergic conjunctivitis, gastrointestinal allergies, cramping, nausea, vomiting, diarrhea, irritable bowel disease, ophthalmic allergies, cheilitis, vulvitis, and anaphylaxis.

18. The method according to claim 17, wherein the pulmonary disease is asthma.

19. A method for the prevention and/or treatment of an allergic disorder mediated by a cell type selected from the group consisting of serosal-type mast cells and basophils, said method comprising the step of administering to a subject in need thereof a pharmaceutical composition comprising a therapeutically effective amount of a peptide and a pharmaceutically acceptable carrier, the peptide selected from the group consisting of:
(SEQ ID NO:15 to 20)
(a) X1-Cys-Asn-R1-X4;
(SEQ ID NO:21 to 23)
(b) X2-Lys-Val-Phe-Leu-Asp-X3;
and
(SEQ ID NO:24)
(c) X5-Asp-Ser-Ser-Asn-Tyr-Ile-R7;
wherein
X1 is selected from hydrogen, lower alkanoyl, Cys, Asp-Cys and Arg-Arg-Cys;
X2 is selected from hydrogen, lower alkanoyl and Lys;
X3 is selected from
(i)
Ala-Ala-Asn-R1-Ile-Thr-R2-Leu-R3-R4;
(ii)
Cys-Cys-Asn-R1-Ile-Thr-R2-Leu-R3;
and
(iii)
Cys-Cys-Asn-R1-Ile-Thr-R2-Leu-R3-R4-Gln-His-R5-R6;
X4 is selected from (i) Ile-Thr-R2-Leu-R3; and (ii) Ile-Thr-Arg-R7;
X5 or a sequence selected from lower alkanoyl and Leu;
R1 is selected from an aromatic amino acid residue;
R2 is selected from Glu and Lys;
R3 is selected from a positively charged amino acid residue;
R4 is selected from Arg and Glu;
R5 is selected from Ala and Arg:
R6 is selected from Arg and Lys;
R7 is selected from hydroxy (OH), Arg, Arg-NH2, and Agm (agmatine),
and analogs, chemical derivatives and pharmaceutically acceptable salts thereof.

20. The method according to claim 19, wherein the peptide selected from the group consisting of:
(SEQ ID NO:25)
(a) Cys-Cys-Asn-Tyr-Ile-Thr-Glu-Leu-Arg;
(SEQ ID NO:26)
(b) Asp-Cys-Cys-Asn-Tyr-Ile-Thr-Arg;
(SEQ ID NO:27)
(c) Asp-Ser-Ser-Asn-Tyr-Ile-Arg;
(SEQ ID NO:28)
(d) Lys-Val-Phe-Leu-Asp-Cys-Cys-Asn-Tyr-Ile-Thr-
Glu-Leu-Arg;
(SEQ ID NO:29)
(e) Lys-Lys-Val-Phe-Leu-Asp-Cys-Cys-Asn-Tyr-Ile-
Thr-Glu-Leu-Arg-Arg-Gln-His-Ala-Arg;
(SEQ ID NO:30)
(f) Lys-Val-Phe-Leu-Asp-Ala-Ala-Asn-Tyr-Ile-Thr-
Glu-Leu-Arg-Arg denoted herein C3a6;
(SEQ ID NO:31)
(g) Arg-Arg-Cys-Cys-Asn-Tyr-Ile-Thr-Arg-Arg
denoted herein C3a10;
(h) an analog of (a), (b), (c), (d), (e), (f) or
(g);
(i) a chemical derivative of (a), (b), (c), (d),
(e), (f), (g) or (h);
(j) a salt of (a), (b), (c), (d), (e), (f), (g),
(h), or (i);
and a pharmaceutically acceptable carrier.

21. The method according to claim 20, wherein the peptide consisting of an amino acid sequence set forth in SEQ ID NO:25 optionally having a carboxy terminal amide.

22. The method according to claim 20, wherein the peptide consisting of an amino acid sequence set forth in SEQ ID NO:26 optionally having a carboxy terminal amide.

23. The method according to claim 19, wherein the allergic disorder resulting from IgE- or IgG-mediated (Type I or Type III) hypersensitivity and/or FcεRI or FcγR-induced secretory response.

24. The method according to claim 19 wherein the allergic disorder is selected from the group consisting of gastrointestinal allergies; cramping; nausea; vomiting; diarrhea; irritable bowel disease.


SEQUENCE LISTING (42 sequences)

SEQ ID NO: 1 - protein, 77 aa, Artificial Sequence
  derived from human sequence
SVQLTEKRMDKVGKYPKELRKCCEDGMRENPMRFSCQRRTRFISLGEACKKVFLDCCNYITELRRQHARASHLGLAR

SEQ ID NO: 2 - protein, 10 aa, Artificial Sequence
  derived from human sequence
DCCNYITELR

SEQ ID NO: 3 - protein, 9 aa, Artificial Sequence
  derived from human sequence
XDSSNYITR

SEQ ID NO: 4 - protein, 11 aa, Artificial Sequence
  derived from human sequence
XDSSNYITECR

SEQ ID NO: 5 - protein, 9 aa, Artificial Sequence
  derived from human sequence
XDVVNYITR

SEQ ID NO: 6 - protein, 11 aa, Artificial Sequence
  derived from human sequence
XDVVNYITECR

SEQ ID NO: 7 - protein, 10 aa, Artificial Sequence
  derived from human sequence
XXDSSNYITR

SEQ ID NO: 8 - protein, 12 aa, Artificial Sequence
  derived from human sequence
XXDSSNYITECR

SEQ ID NO: 9 - protein, 10 aa, Artificial Sequence
  derived from human sequence
XXDVVNYITR

SEQ ID NO: 10 - protein, 12 aa, Artificial Sequence
  derived from human sequence
XXDVVNYITECR

SEQ ID NO: 11 - protein, 9 aa, Artificial Sequence
  derived from human sequence
SDDSNYITR

SEQ ID NO: 12 - protein, 8 aa, Artificial Sequence
  derived from human sequence
DVVNYITR

SEQ ID NO: 13 - protein, 9 aa, Artificial Sequence
  derived from human sequence
CDSSNYITR

SEQ ID NO: 14 - protein, 11 aa, Artificial Sequence
  derived from human sequence
SDSSNYITECR

SEQ ID NO: 15 - protein, 9 aa, Artificial Sequence
  derived from human sequence
XCNXITXLX

SEQ ID NO: 16 - protein, 8 aa, Artificial Sequence
  derived from human sequence
XCNXITRX

SEQ ID NO: 17 - protein, 10 aa, Artificial Sequence
  derived from human sequence
DCCNXITXLX

SEQ ID NO: 18 - protein, 9 aa, Artificial Sequence
  derived from human sequence
DCCNXITRX

SEQ ID NO: 19 - protein, 11 aa, Artificial Sequence
  derived from human sequence
RRCCNXITXLX

SEQ ID NO: 20 - protein, 10 aa, Artificial Sequence
  derived from human sequence
RRCCNXITRX

SEQ ID NO: 21 - protein, 16 aa, Artificial Sequence
  derived from human sequence
XKVFLDAANXITXLXX

SEQ ID NO: 22 - protein, 15 aa, Artificial Sequence
  derived from human sequence
XKVFLDCCNXITXLX

SEQ ID NO: 23 - protein, 20 aa, Artificial Sequence
  derived from human sequence
XKVFLDCCNXITXLXXQHXX

SEQ ID NO: 24 - protein, 8 aa, Artificial Sequence
  derived from human sequence
XDSSNYIX

SEQ ID NO: 25 - protein, 9 aa, Artificial Sequence
  derived from human sequence
CCNYITELR

SEQ ID NO: 26 - protein, 8 aa, Artificial Sequence
  derived from human sequence
DCCNYITR

SEQ ID NO: 27 - protein, 7 aa, Artificial Sequence
  derived from human sequence
DSSNYIR

SEQ ID NO: 28 - protein, 14 aa, Artificial Sequence
  derived from human sequence
KVFLDCCNYITELR

SEQ ID NO: 29 - protein, 20 aa, Artificial Sequence
  derived from human sequence
KKVFLDCCNYITELRRQHAR

SEQ ID NO: 30 - protein, 15 aa, Artificial Sequence
  derived from human sequence
KVFLDAANYITELRR

SEQ ID NO: 31 - protein, 10 aa, Artificial Sequence
  derived from human sequence
RRCCNYITRR

SEQ ID NO: 32 - protein, 8 aa, Artificial Sequence
  derived from human sequence
DSSNYITR

SEQ ID NO: 33 - protein, 20 aa, Artificial Sequence
  derived from human sequence
SVQLTEKRMDKVGKYPKELR

SEQ ID NO: 34 - protein, 13 aa, Artificial Sequence
  derived from human sequence
RQHARASHLGLAR

SEQ ID NO: 35 - protein, 9 aa, Artificial Sequence
  derived from human sequence
RTIYNSSDS

SEQ ID NO: 36 - protein, 9 aa, Artificial Sequence
  derived from human sequence
GAKDGNEYI

SEQ ID NO: 37 - protein, 5 aa, Artificial Sequence
  derived from human sequence
CCNYG

SEQ ID NO: 38 - protein, 8 aa, Artificial Sequence
  derived from human sequence
DVSNYITR

SEQ ID NO: 39 - protein, 22 aa, Rattus norvegicus
STLQTSDFDDEVLLLYRAGYPF

SEQ ID NO: 40 - protein, 20 aa, Homo sapiens
SVLDISHIEGDIFSSFKAGY

SEQ ID NO: 41 - protein, 22 aa, Rattus norvegicus
NNSAYMNYCKDITEDDGCFVTS

SEQ ID NO: 42 - protein, 23 aa, Homo sapiens
KSLAYIHIHSCQKFFETKCFMAS